(19) United States
(12) Patent Application Publication
(10) Pub. No.: US 2013/0130931 A1
(43) Pub. Date: May 23, 2013

(54) BIOMARKER FOR THE DIAGNOSIS, PROGNOSIS AND MONITORING OF CANCER

(71) Applicant: CONSEJO SUPERIOR DE INVESTIGACIONES CIENTIFICAS (CSIC), Madrid (ES)

(72) Inventor: Ana Clara CARRERA RAMIREZ, Madrid (ES)

(73) Assignee: CONSEJO SUPERIOR DE INVESTIGACIONES CIENTIFICAS (CSIC), Madrid (ES)

(21) Appl. No.: 13/746,619

(22) Filed: Jan. 22, 2013

Related U.S. Application Data
(63) Continuation of application No. PCT/ES2011/070451, filed on Jun. 21, 2011.

(30) Foreign Application Priority Data
Jul. 22, 2010 (ES) P201031137

(51) Int. Cl. C12Q 1/68; G01N 33/68

(52) U.S. Cl. CPC C12Q 1/6886; G01N 33/6893
USPC 506/9; 435/7.4; 435/6.14; 536/24.5; 506/16

(57) ABSTRACT

The present invention provides a new biomarker for the diagnosis, prognosis and monitoring of cancer, preferably breast and colon cancer, the PIK3R2 gene or any of its expression products; since high levels of PIK3R2 correlate with advanced tumor stages, and in the case of breast cancer, with invasive or metastatic capacity. Thus, the invention is also related to a method for the diagnosis, prognosis and monitoring of cancer, preferably breast or colon cancer, in which the quantification of said biomarker in a biological sample comprising tumor cells is necessary.

The present invention falls within the field of Oncology, specifically, within the biomarkers useful for the diagnosis, prognosis and monitoring of malignant tumors, preferably breast and colon cancer, and within the methods for the diagnosis, prognosis and monitoring of said tumors in which the quantification of such biomarkers is necessary. The invention is also related to the use of compounds inhibitors of these biomarkers for cancer treatment, preferably, colon and breast cancer.

### STATE OF THE PRIOR ART

In cancer, prognostic and predictive factors are useful in medical decision-making about the management and treatment of the disease. Thus, e.g., in the case of breast cancer, the following are considered validated as prognostic factors: the status of axillary nodes, tumor size, histological type and grade, age of the patient, and other factors such as biomarkers measurable in tissues, cells and fluids, such as the status of steroid receptors (RE-RP). In the latter group, c-erbB-2 over expression, the status of p53, the histological evidence of vascular invasion and the angiogenesis quantitative parameters, have been widely studied, clinically and biologically, but they do not have an established role in the management of patients, which is why these parameters are not sufficient to predict the course of the disease.

Phosphoinositide 3-kinases (PI3K) are lipid enzymes that phosphorylate the 3D position of the inositol ring of membrane phosphoinositides (PtdIns) to generate PtdIns(3,4)P₂ (PIP₂) and PtdIns(3,4,5)P₃ (PIP₃), which are capable of activating pathways such as the protein kinase B (PKB or Akt) pathway that induce cell division and survival (Kok K., et al., 2009, Trends Biochem. Sci. 34: 115-127).

PI3K are heterodimers formed by a p85 regulatory subunit and a p110 catalytic subunit. There are four catalytic subunits encoded by the genes PIK3CA, CB, CD and CG. p110 g (PIK3CG) is associated to the p87 or p101 regulatory subunits and is activated by receptors associated with G proteins, while the other catalytic subunits are associated with p85-type regulatory subunits and are mainly activated by receptors with tyrosine kinase activity. p85 regulates the stability of p110 and mediates its translocation to the membrane and its activation after stimulation of growth factor receptors. There are three genes that encode p85-type subunits, the ubiquitous genes PIK3R1 (p85α) and PIK3R2 (p85β), and the tissue specific gene PIK3R3 (p55γ) (Yuan T. L. and Cantley L. C., 2008, Proc. Natl. Acad. Sci. USA 105: 9739-9744).

PI3K regulates the division, migration and cell survival and it is known that mutations that induce aberrant activation of this pathway, including the deletion of the negative regulator, PTEN phosphatase, PIK3CA activating mutations and increased expression of PIK3CB or PIK3CD, are common in cancer. Over expression and mutation of genes encoding the p110 catalytic subunits in cancer have been studied extensively (Cully M., et al., 2006, Nat Rev Cancer 6: 184-192). The PIK3R1 (p85α) gene has been identified as the oncogene involved in tumor processes in colon and ovary (Amanda J. Philp, et al., 2001. Cancer Research 61: 7426-7429). Likewise, an analysis of the expression profile of the mRNA of PI3K genes in different types of tumors (Lin Zhang, et al., 2007, Clinical Cancer Research 13(18): 5314-5321) showed that 6 of them show an increase in their number of copies in the ovary tumor cells, as well as in other types of cancers, such as colon or breast cancer, compared to the corresponding normal tissues. In this respect, the PIK3R3 gene has been proposed as a therapeutic target, being the gene with the most significant over expression in the analyzed tumors.

Since the PI3K activity is increased in hyperproliferative processes, this enzyme has been proposed as a therapeutic target in cancer. In this respect, e.g., perifosine has inhibitory effects on tumor cells in breast cancer, among other types of cancer, through inhibition of the PI3K signaling pathway (Bryan T. Hennessy, et al., 2007, Clinical Cancer Research 13(24): 7421-7431).

However, there is still the need to find new biomarkers that can be used to diagnose, not only the absence or presence of tumors, but advanced tumor stages in cancer patients. In this way, disease monitoring could be made easier and predictive assessments on the course of the same could be established as e.g., invasive capacity of the tumor, and thus, guiding the medical decisions regarding the most appropriate treatment to be administered in each particular clinical case.

### DESCRIPTION OF THE INVENTION

The present invention provides a new biomarker for the diagnosis, prognosis and monitoring of cancer, PIK3R2 gene or any of its expression products, as well as a method for the diagnosis, prognosis and monitoring of this disease, preferably breast or colon cancer, comprising its quantification in a biological sample containing tumor cells.

As the examples of the present invention show, tumors, preferably breast and colon cancer, have a change in the PI3K regulatory subunit used increasing the expression of p85β (PIK3R2) and reducing the expression of p85α (PIK3R1). This change to p85β correlates with tumor progression, i.e. malignant tumor phenotypes have high expression levels of this gene in comparison with non tumor tissues. In the case of colon carcinomas, the expression of PIK3R2 is related to tumor grade and in the case of breast cancer, the expression levels of PIK3R2 correlate with invasive or metastatic capacity. Thus, the present invention demonstrates that the analysis of the expression of the biomarker PIK3R2 is a method useful for the diagnosis, prognosis and monitoring of tumor progression.

For this reason, one aspect of the invention is related to the use of the PIK3R2 gene or its expression products for the diagnosis, prognosis and monitoring of cancer. In a preferred embodiment, the cancer is breast or colon cancer.

“Diagnosis” means the procedure through which the degree or stage of a disease, preferably neoplastic, is identified. In the context of the present invention, it is related to the identification of an advanced stage in a tumor of an individual or the identification of an invasive or metastatic tumor. The term “prognosis” is related to the procedure whereby a prediction of events which will occur in the development or course of a disease, preferably neoplastic, is established, including relapse, capacity of metastatic dissemination or response to a certain treatment.

The coding nucleotide sequence of the PIK3R2 gene is the sequence from GenBank reference number NM_{—}005027. The term “expression product”, such and as used in this description refers to any transcription product (RNA, including forms of alternative rearrangement) or expression (protein) of this gene, or to any form resulting from the processing of said transcription or expression products. The expression products of this gene are, preferably, mRNA coding the protein p85β or protein p85β.

The term “cancer”, such and as used in the present description refers to the neoplastic disease in which cells with an abnormal morphology have uncontrolled growth generating a tumor. Examples of cancer include, but are not limited to, hepatocellular carcinoma or liver cancer, prostate cancer, lung cancer, pancreatic cancer, colon cancer, breast cancer, gynecological cancers, such as ovarian, uterus, cervix, vagina or vulva, skin cancer, such as melanoma, esophageal cancer, stomach cancer, bladder cancer, cancer of the urinary tract, thyroid cancer, renal cancer, brain cancer, sarcoma, lymphoma or leukemia.

“Breast cancer” refers to the type of malignant neoplasm that has its origin in the accelerated and uncontrolled proliferation of cells that cover the inside of the ducts that carry milk from the glandular acinus to the milk ducts located behind the areola and the nipple during breastfeeding, or the type of malignant neoplasm that originates in the glandular acinus. This term includes, without limitation, adenocarcinoma, located in the glandular tissue; cystosarcoma; sarcoma; ductal carcinoma, located in the breast or milk ducts; lobular carcinoma which includes lobular neoplasia; inflammatory breast cancer, where the cancer cells block the lymphatic vessels, which manifests on the skin that acquires a thick and recessed appearance; Paget's disease, which spreads through the skin of the nipple and the areola, which appear scaly and reddish; mucinous or colloid cancer, in which cancer cells produce mucus; or Medullary cancer, an infiltrating tumor. Breast cancer can be detected using techniques known in the biomedical field such as e.g., but not limited to, mammography, magnetic resonance imaging, ultrasonography or biopsy.

“Colorectal cancer” or “colon cancer”, includes any type of cancer of the colon, rectum or appendix. Colon cancer can be detected by using, e.g., but not limited to, rectal examination, fecal occult blood test (FOBT), sigmoidoscopy, colonoscopy, virtual colonoscopy, double contrast barium enema, ultrasound, biopsy or nuclear magnetic resonance (NMR).

Another aspect of the invention relates to a method for obtaining data useful for the diagnosis, prognosis and monitoring of cancer, hereinafter “method of the invention”, comprising:
a. obtaining an isolated biological sample comprising tumor cells from an individual,
b. detecting the amount of expression product of the PIK3R2 gene in the isolated biological sample from (a), and
c. comparing the amount detected in step (b) with a reference amount.

The term “isolated biological sample comprising tumor cells”, such as used in the description is related, but not limited to, to tissues and/or biological fluids from an individual obtained through any method known by a person skilled in the art useful for this purpose. The biological sample may be a tissue, e.g., but not limited to, tumor biopsy or fine needle aspirate, or it can be a biological fluid, e.g., but not limited to, a sample of fluid, such as blood, plasma, serum, lymph, ascitic fluid, urine or breast exudates. The sample can be taken from a human, but also from non-human mammals, such as e.g., but not limited to, rodents, ruminants, feline or canine. Therefore, in a preferred embodiment of this aspect of the invention, the individual from whom the isolated biological sample of step (a) of the method of the invention proceeds is a mammal. In a more preferred embodiment, the mammal is a human.

The detection of the amount of expression product of the PIK3R2 gene in the sample obtained, refers to the measurement of the amount or concentration, preferably in quantitative or semi quantitative way. This measurement can be carried out in a direct or indirect way. The direct measurement refers to the measurement of the amount or concentration of the gene expression product based on a signal obtained directly from the gene expression product and which is directly correlated with the number of molecules of the gene expression product present in the sample. Said signal—which can also be referred to as intensity signal—may be obtained, e.g., by measuring an intensity value of a chemical or physical property of the expression product. The indirect measurement includes measurements obtained from a secondary component (e.g., a component other than the gene expression product) or a system of biological measurement (e.g., the measurement of cellular responses, ligands, ‘tags’ or enzymatic reaction products).

According to the present invention, the detection of the amount of gene expression product can be carried out by any method for the determination of the amount of the gene expression products known by the person skilled in the art. In a preferred embodiment, the detection of the amount of gene expression product is made by determining the mRNA level derived from its transcription, after extraction of the total RNA from the isolated biological sample which can be carried out by methods known by a person skilled in the art. The analysis of the level of mRNA can be performed, for illustrative purposes and without limiting the scope of the invention, through amplification by polymerase chain reaction (PCR), retro-transcription in combination with the ligase chain reaction (RTLCR), retro-transcription in combination with polymerase chain reaction (RT-PCR), retro-transcription in combination with quantitative polymerase chain reaction (qRT-PCR), or any other method of nucleic acid amplification; DNA microarrays made with oligonucleotides deposited by any mechanism; DNA microarrays made with oligonucleotides synthesized in situ using photolithography or by any other mechanism; hybridization in situ using specific probes marked with any marking method; by means of electrophoresis gels; by transfer to membrane and hybridization with a specific probe; using MRI or any other imaging techniques using paramagnetic nanoparticles or any other type of detectable nanoparticles functionalized with antibodies or by any other means. In another preferred embodiment, the detection of the amount of gene expression product is carried out by determining the level of protein p85β, by using, e.g., but not limited to, immunohistochemistry or western blot. Finally, since the translation ability of PIK3R2 to protein p85β is inhibited by the microRNA-126, another preferred embodiment includes, as indirect method for the determination of the levels of p85β, the measurement of the expression levels of the microRNA-126.

The term “reference amount”, such and as used in the present description refers to any value or range of values derived from the quantification of the expression product of the PIK3R2 gene in a control biological sample. In a preferred embodiment, the reference amount comes from an isolated biological sample that does not comprise tumor cells (control biological sample), where said sample may proceed from the individual of step (a) or another individual.

In another preferred embodiment, the method of the invention further comprises:
d. assigning the individual from step (a) to the group of patients with advanced tumor stage when the amount detected in step (b) is greater than the reference amount.

Such and as used in the present description, the term “advanced tumor stage” refers to the tumor stage or phase in which neoplastic cells or cells with uncontrolled growth have reached deep layers of tissue, and that can lead or not to metastasis.

In the case of, e.g., but not limited to, colon cancer, the samples with normal expression levels of PIK3R2 (or p85β), i.e., that do not exceed the reference amount, correlate with low grades of tumor development, as e.g. but not limited to, grades from 0 to A according to the Duke's stratification scale. However, samples with a high content of expression product of the PIK3R2 gene, i.e., with a level of expression of this gene exceeding the reference amount, have a greater tumor grade (grades from A, i.e., B, C or D) than those samples with normal expression levels of this gene. In this respect, the greater the amount of expression product of the PIK3R2 gene in the biological sample analyzed, the more advanced is the tumor stage of the individual from whom the sample proceeds. Therefore, in a more preferred embodiment of this aspect of the invention, the cancer is colon cancer.

Sometimes it may happen that cells within an advanced stage tumor have an invasive or metastatic phenotype. The term “invasive cancer”, such as it appears in the present invention, is related to the type of cancer in which cells within the tumor possess ability to metastasize to other tissues.

In a more preferred embodiment, the cancer is breast cancer. In the case of this type of cancer, the samples that have high expression levels of the PIK3R2 gene (or p85β), i.e., expression levels of this gene/protein exceeding the reference amount, come from regions around which there is a high percentage of lymph nodules which have tumor cells. This means that the samples with expression levels of this gene above the reference amount have cells with higher invasive capacity than the cells from samples with normal expression levels of this gene. Therefore, in a still more preferred embodiment, when the cancer is breast cancer, the method of the invention comprising steps (a) to (c) further comprises:
d. assigning the individual from step (a) to the group of patients with invasive cancer when the amount detected in step (b) is greater than the reference amount.

Steps (b) and/or (c) of the method of the invention can be total or partially automated, e.g., but not limited to, using robotic equipment for the detection, in step (b), of the amount of the PIK3R2 gene or of the expression product of the PIK3R2 gene in the isolated biological sample.

In addition to the steps specified above, the method of the invention may include other additional steps, e.g., but without limitation, relating to pre-treatment of the isolated biological sample before its analysis.

Therefore, the method of the invention is useful for establishing a diagnosis (stage), prognosis and monitoring of cancer, detecting the presence of hidden metastases and recurrences, monitoring the response to treatment and even for performing samplings in the population.

Another aspect of the invention is related to a kit for the diagnosis, prognosis and monitoring of cancer, hereinafter “kit of the invention”, comprising the primers, probes or antibodies, or any combination thereof, necessary for detecting the amount of expression product of the PIK3R2 gene in an isolated biological sample.

The primers, probes and/or antibodies comprised in the kit of the invention have complementarity, and therefore hybridization ability, with at least one expression product of the PIK3R2 gene. Preferably, the kit of the invention further comprises primers, probes or antibodies, or any combination thereof, complementary to the microRNA-126. In general, the kit of the invention comprises all the reagents necessary for carrying out the method of the invention described above. The kit also may include, without any kind of limitation, buffers, enzymes, such as e.g., but not limited to, polymerases, cofactors for obtaining an optimum activity of these, agents to prevent contamination, etc. On the other hand the kit may include all the supports and containers necessary for its implementation and optimization. The kit may contain also other molecules, genes, proteins or probes of interest, which serve as positive and negative controls. Preferably, the kit includes also the instructions to carry out the method of the invention.

Another aspect of the invention is related to the use of the kit of the invention for the diagnosis, prognosis and monitoring of cancer. In a preferred embodiment, the cancer is breast or colon cancer.

On the other hand, when p85β is associated with the catalytic subunit p110α, the heterodimer formed has a greater affinity for the physiological substrate present in the cell membrane, resulting in an increase of the PI3K activity and tumor progression. Therefore, another aspect of the invention is related to the use of at least one inhibitor of the PIK3R2 gene or its expression products for the preparation of a medicament for cancer treatment. In a preferred embodiment, the cancer is breast or colon cancer. In a more preferred embodiment, the cancer is breast cancer.

In the present invention “inhibitor” means any compound that allows to reduce, block or delete the expression of the PIK3R2 gene or the transport or activity of any its expression products. Examples of inhibitors of this type are, but without limitation, siRNAs or microRNAs complementary to the mRNA resulting from the transcription of this gene; or p85β protein inhibitors such as, e.g., but not limited to: peptides that relocate p85β or that displace its association with p110.

Throughout the description and the claims the word “comprises” and its variants are not intended to exclude other technical features, additives, components or steps. For those skilled in the art, other objects, features and advantages of the invention will derive in part from the description and in part from the practice of the invention. The following examples and drawings are provided by way of illustration, and are not intended to be limiting of the present invention.

### DESCRIPTION OF THE FIGURES

FIG. 1 shows the level of expression, by Q-PCR, of p85β (PIK3R2, R2) (x-axis) and p85α (PIK3R1, R1) (y-axis) in breast (BC) and colon (CC) carcinomas. n=number of samples. Inactive: samples with inactive PIK3 activity. Active: samples with PIK3 activity. The values of R2 are equal or higher than in adjacent normal tissue. The values of R1 in the CC are equal to (normalized R1=0) or lower (R1<0) than in normal tissue; the values of R1 in the BC are lower (normalized R1 between −2 and −3); or show small changes (R1 between −1 and +1) with respect to normal tissue. (*) Chi-square, P=0.016 (BC) and 0.012 (CC).

FIG. 2 shows that the increase in the expression of p85β correlates with tumor progression. (a) p85α and p85β levels were tested in Western Blot (WB) in different samples of BC and CC. The graphs show the signal intensity of p85α and p85β (indicated as R1 and R2) in arbitrary units (A.U.) normalized against Actin. The arrows indicate the normal expression levels of p85α and p85β. The normalized R2 and R1 mRNA values (analyzed by Q-PCR) are shown at the bottom. (b) Percentage of CC with equal (equal) or higher (high) levels of PIK3R2 (R2) measured by Q-PCR in tumor compared with normal tissue. Tumors were grouped according to the degree of activity of PI3K (active/inactive). (*) Chi-square value for these data P=0.02. (c, d) Tumor grade in samples of colon carcinoma (CC) with active or inactive PI3K (c), or in the complete collection of tumors (d) depicted against the levels of expression of R2 by Q-PCR. 0-A grade CC are depicted with those of grade 0. (**) Chi-square P=0.001 (c) and 0.002 (d). (e) Percentage of breast cancers (BC) according to the expression levels of R2 measured by Q-PCR. (*) Chi-square P=0.04. (f) Tumor grade in BC with active or inactive PI3K in relation to the levels of R2 by Q-PCR. n.s.; not significant. (g) BC invasion/metastasis determined as a percentage of lymph nodes (LN) with infiltrated tumor cells with respect to total LN, depicted against the levels of R2. (**) Pearson test P=0.002; samples with very high R2 (≧2), high R2 (1) or R2 equal to the normal adjacent tissue (0) are compared. (***) Pearson P=0.0008.

FIG. 3 shows the increase in the activation of the PI3K pathway in cells that express p85β. (a) COS-7 cells were transfected with pSG5-p110α combined with pSG5-p85α or -p85β (24 hrs), then they were incubated (1) in serum-free medium (48 hrs); (2) some samples were subsequently activated with serum (10%) or (3) PDGF (50 ng/ml) for 10 min. Exponential: COS-7 cells in exponential growth. Control: non-transfected COS-7 cells. It shows the protein expression levels and activation of the PI3K effectors (lower gels) tested by WB, using Actin as a control. (b) It shows the quantification of the signal of the PIK3 effectors pPKB and pp70S6K from three experiments in control and transfected cells (mean±Standard deviation, n=3; AU). (c) HeLa cells were transfected with p85β siRNA (100 nM, β1 and 200 nM, β2), (24 and 48 hrs); extracts were prepared and assayed by WB to study the efficiency of the siRNA in the reduction of the p85β levels, as well as phosphorylation of the PKB and p70S6k effectors, using Actin as a control. Ctr: control cells extracts, transfected with control siRNA. Graphs as in (b). (*) Student's t test P<0.01.

FIG. 4 shows that p85β increases the content of PIP₃ in the membrane and induces cell transformation. (a) NIH3T3 cells were transfected with GFP-Btk-PH in combination with p85α/p110α, or p85β/p110α (24 hrs), they were incubated (19 hrs) in serum-free medium (quiescence) or activated with serum (15 min; right). Control: non-transfected cells. The location of PIP₃ (GFP-Btk-PH) was tested by fluorescence microscopy. The arrows indicate the sections in which the fluorescence signal was measured (AU). (b) Percentage of cells with the PIP₃ signal concentrated in the membrane, cytosol or both (intermediate), and of cells with disc-shaped, mesenchymal, or intermediate morphology. (c) Assays of formation of representative foci in NIH3T3 control cells and in cells transfected with p85α, p85β or V12-Ras (mean±Standard deviation, n=6). (**) Student's t test P<0.001. Bar=15 μm.

### EXAMPLES

Next, the invention will be illustrated by means of tests performed by the inventors, which highlight the specificity and effectiveness of the method of the invention in the diagnosis, prognosis and monitoring of cancer, preferably breast and colon cancer. These specific examples provided serve to illustrate the nature of the present invention and are included only for illustrative purposes, so they should not be construed as limitations to the invention claimed here. Therefore, the examples described below illustrate the invention without limiting the field of application of the same.

### Example 1

### Alterations in the PI3K Pathway in Colon and Breast Clinical Tumors

The status of the PI3K pathway was tested in a collection of colon adenocarcinomas (CC) and breast ductal carcinomas (BC) comparing them to the status of normal adjacent tissue. A protocol was designed to analyze these samples that included immunohistochemistry (IH) analysis, quantitative PCR (Q-PCR) and Western blot (WB) analysis.

The samples of both breast (BC) and colon (CC) carcinoma and the adjacent normal tissue samples were obtained from the collection of tumors of the Centro Nacional de Investigaciones Oncológicas (CNIO, Madrid, Spain).

The immunohistochemistry intensity signals were in a range from 1 to 3 (1, low level of staining; 2 was ≧3 times higher than the background; 3 was ≧6 times higher than the background). For the WB analysis, frozen sections of the samples were lysed in RIPA buffer; the WB signal was quantified and normalized by loading Actin controls. Said signals were measured over a range from −2 to +2 (0 indicates no changes, ±1 reflects an increase or decrease between 20% and 50%; and ±2 changes above 50%).

For the analysis by Q-PCR, the mRNA was obtained from the frozen samples and it was analyzed in custom-designed TaqMan Low Density Arrays (LDA, Applied Biosystems) containing the primers and probes of the PI3K genes and f their regulators. As template RNA for reverse transcription, 1 ng of total RNA per sample was used (triplicate).

Q-PCR was carried out in an ABI PRISM 7900 HT (Applied Biosystems). The LDA probes used were GAPDH (probe Hs4342376), ACTB (Hs99999903), AKT1 (Hs00178289), AKT2 (Hs00609846), AKT3 (Hs00178533), IGF1 (Hs00153126), IGF1R (Hs00609566), IGF2 (Hs00171254), IGFBP3 (Hs00426287), SHIP1 (Hs00183290), PIK3CA (Hs00180679), PIK3CB (Hs00178872), PIK3CD (Hs00192399), PIK3CG (Hs00176916), PIK3R1 (Hs00236128), PIK3R2 (Hs00178181), PIK3R3 (Hs00177524) and PTEN (Hs00829813). For PIK3CG and PIK3R1, Hs00277090 and Hs00381459 were also used.

Relative quantification of mRNA was determined by the ΔΔCt method. 2^{-ΔΔCt} Values (<1 represents decrease and >1 represents increase). To facilitate comparison, the different values were normalized in a range from −3 to +3 (where 0 indicates no changes or insignificant changes (2^{-ΔΔCt} between 0.6-1.2); 1-3 indicates increase, 1 (2^{-ΔΔCt} 1.2-3.0), 2 (2^{-ΔΔCt} 3.0-6.0), 3 (2^{-ΔΔCt}>6); and negative values indicate a decrease −1 (2^{-ΔΔCt} 0.6-0.3), −2 (2^{-ΔΔCt} 0.3-0.1), −3 (2^{-ΔΔCt}<0.1)).

The primary antibodies used for the WB were anti-phospho-(p)-PKB (Ser473), -p-p70s6k (Thr389), -p-PKC (zeta-Thr410) from Cell Signaling; anti-pan-p85 PI3K, -human p85α and -PKB from Upstate Biotechnology; -p70S6K (C-18) and -PTEN (N-19) from Santa Cruz Biotechnology. Anti-HA (12CA5) was from Babco and anti-β-Actin from Sigma-Aldrich. The Anti-p110α was donated by A. Klippel. Anti-p85β PI3K (rat Ab 1C8,) and anti-HA (12CA5) antibodies were used for immunoprecipitation (IP). For the anti-p85β antibodies, mice were immunized with the C-terminal KLH-conjugated peptide (residues 711-722 of p85β) or rats were immunized with a N-terminal GST-fused fragment. Anti-p85β-specific antibody was tested in ELISA, WB and IP using recombinant proteins from bacteria or cells expressing r-p85β or r-p85α extracts. Rabbit K1123 antibody strongly recognized p85β in WB and slightly recognized p85α. Rat monoclonal antibody (mAb) 1C8 was efficient in recognition of p85β by WB and IP, but it did not recognize p85α; this mAb was affinity-purified (GST kit; Pierce).

Immunoprecipitation was carried out as described (Marqués M., et al., 2008, Mol Cell Biol., 28: 2803-2814). For the WB, cells were lysed in 1% Triton X-100 (TX-100) medium containing protease and phosphatase inhibitors. Human tumor lines were lysed in RIPA medium containing protease inhibitors and phosphatase inhibitors.

For IH, the first 10 samples were analyzed using anti-phospho-PKB (P-PKB) antibodies (Ab), however, anti-phospho-S6 antibodies (p-S6; Cell Signaling) gave a better and more consistent signal, and therefore they were used for the rest of the samples. To examine the levels of mRNA of the members of the PI3K pathway, the aforementioned TaqMan cards (which will be called PIP-chip) were used; the above described antibodies which recognize different PI3K catalytic and regulator subunits and their regulators were used for WB. The entire analysis was carried out in 95% of the CC and in 85% of the BC.

For statistical analysis, the relationship between the tumor variables was assayed by the Pearson test and the contingency of the parameters of the tumors by the Chi-square test were calculated using Prism5V.5.0b software. The bands of gel and the intensity of the fluorescence were quantified by ImageJ software. The Student's t test was carried out using StatView 512+.

IH showed that the majority of the samples were heterogeneous, since only a fraction of the tumor cells in a particular tumor was positive for p-S6. Samples in which the positivity ratio for p-S6 or p-PKB was higher than 50%, or samples when 30% to 40% of cells were highly positive (3 on a scale of 1 to 3) were classified as active samples. PI3K activity was also tested by WB using anti-pPKB and -pp70S6K Ab; approximately 80% of the carcinoma samples that were positive by IH were also positive by WB. The positivity by WB was not detected only when the ratio of positive cells in a tumor was very low. IH and WB analysis showed that a third of the BC samples and 55% of the CC samples had the PI3K pathway activated, which results were inside the range of previous studies. This pathway activity correlated with the tumor stage in CC (in accordance with the criterion of Dukes, stratification A to D) (DeVita V. T., et al., 2005, Philadelphia USA. P. 1239-1242). For breast carcinoma (BC, Bloom Richardson grades I to III) (Bloom M. J., et al., 1962, Br Med J. 5299, 213), the correlation was not statistically significant although advanced tumors (grade II/III or III) had frequently active PI3K, however, in these tumors there was a correlation of p85β levels with invasiveness as shown below.

It was evaluated if the activation state of the PI3K pathway correlated with changes in the levels of mRNA of the main regulators of the PI3K pathway. Using PIP-chip, the levels of the p110 catalytic subunits, the p85 regulatory subunits, SHIP1, PTEN and the PKB isoforms were measured, as well as the levels of the elements of the IGF pathway. This assay was complemented by the assessment by WB of the protein levels of the majority of these molecules in 10 samples, approximately. The alteration of the mRNA that encodes PTEN, p110α, β, or δ, p85α or β, PKBβ (AKT2) and SHIP1 was consistent with changes in the expression of the corresponding proteins by WB (in 75% or more of the cases examined). Post-transcriptional or post-translational regulation could explain the lack of correlation in 100% of the cases.

The PIP-chip and WB analyses detected that in a high proportion of BC and CC samples with inactive PI3K, the PTEN protein had a high expression; the difference in the expression of PTEN among the samples with active or inactive PI3K was significant in the case of BC (n=22).

In addition to the PIK3CA mutation, previously observed in several types of tumors, the expression of the PIK3CB mRNA was increased in 25% of the CC and in approximately 15% of the BC, while the expression of PIK3CD mRNA was increased in 20% of the BC and CC; these expression changes were confirmed by WB in approximately 10 samples of tumors of each type. PIK3CG was also increased in 25% of the BC, but since the WB showed very low levels of protein, it is not clear whether this change contributes to the behavior of the tumor. Approximately 30% of the CC and 50% of the BC also showed an increase in the levels of AKT2, although this was not correlated with the activation of the pathway, tumor stage or invasion of lymph nodes.

Expression patterns characteristic of tumor type were also observed. Q-PCR PIP-chip showed that 60% of the CC, and only 8% of the BC, showed an increase in the expression of SHIP1, a phenotype that was mainly found in active samples in PI3K. The IGF pathway was frequently altered in BC (55%) and CC (85%), with increases in the expression of IGF1R, more frequently in BC, and increase in the levels of IGF2, more frequently in CC. However, the most striking observation was the change in the expression of the PI3K ubiquitous regulatory subunits of class IA.

### Example 2

### An Increase in the Levels of p85β in Colon and Breast Carcinoma Correlates with Tumor Progression

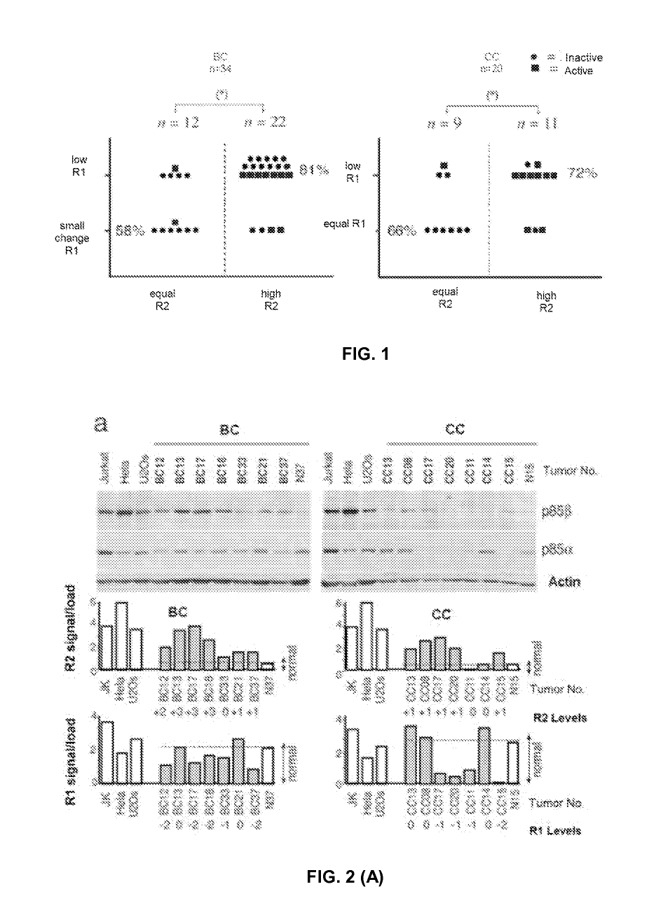

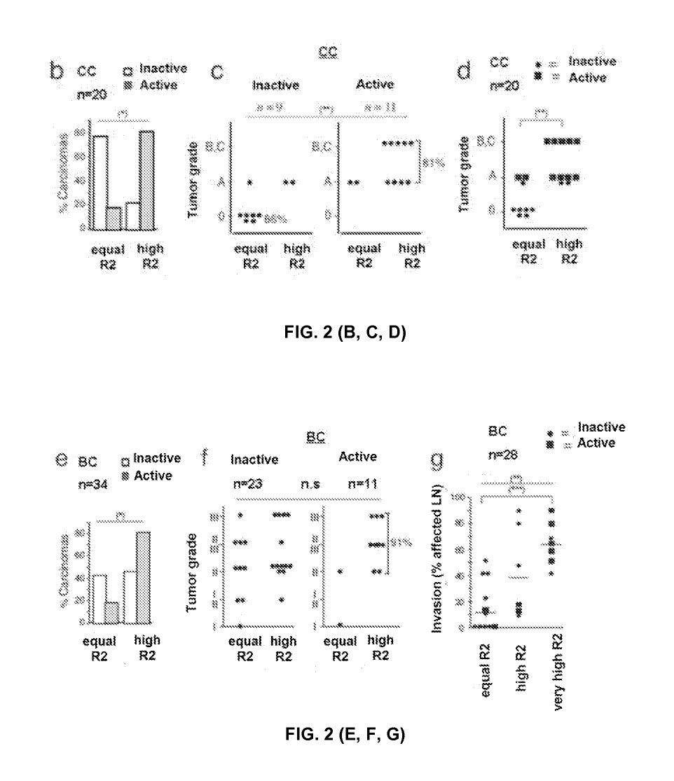

In normal tissues p85α (PIK3R1) is usually expressed at higher levels than p85β (PIK3R2). The analyses normalized by Q-PCR on PIP-chip cards showed that >50% of colon and breast carcinomas showed an increase in the expression of PIK3R2 (p85β). In addition, the increase in the levels of PIK3R2 was found in samples with a decrease in the levels of PIK3R1 (p85α) (FIG. 1). The increase of PIK3R2 by Q-PCR was confirmed in northern blot. Antibodies specific for p85 were prepared and the increases of p85β expression in CC and BC were confirmed by WB (FIG. 2a).

Next, it was assessed whether the increase in p85β expression correlated with the activation of the PI3K pathway in CC. It was found that the samples with increased levels of PIK3R2 mRNA were found more often in CC samples with active PI3K (FIG. 2b). In addition, while the tumors with normal PIK3R2 levels had grades from 0 to 0-A, the samples with high content in PIK3R2 had higher grade (FIG. 2c). In fact, the expression of PIK3R2 correlated with tumor grade (FIG. 2d). Therefore, the PIK3R2 expression level can be considered a biomarker of tumor progression in CC.

While in CC Duke's stratification describes the penetration of the tumor cells in the tissue layers, in BC the criterion of Bloom Richardson (BR) describes cell differentiation, and to evaluate the invasiveness it is reported the proportion of lymph nodes (LN) having tumor cells in tumor surroundings. In BC, although the high levels of PIK3R2 were more frequently found in tumors with active PI3K, the levels of PIK3R2 mRNA were also increased in approximately 40% of samples with inactive PI3K (FIG. 2e). The increase in the levels of PIK3R2 did not correlate with Bloom Richardson grade (FIG. 2f), although malignant phenotypes had high levels of PIK3R2 in active samples (FIG. 2f). The possible correlation between the levels of PIK3R2 mRNA and invasive/metastatic phenotype was assessed. The invasive potential was quantified as a percentage of LN infiltrated by tumor cells; thus, the levels of PIK3R2 correlated with metastatic ability (FIG. 2g).

Thus, the comparison of the expression levels of the PI3K regulatory and catalytic subunits showed that the mRNA and the protein p85β (PIK3R2) increase in these tumors (>50%). The increase of p85β in colon carcinomas correlated with tumor grade. Therefore, in colon, the measure of the levels of PIK3R2 can be considered a biomarker of tumor progression. In breast, while the levels of p85β increase in samples with active and inactive PI3K, they were more often high in those active. In breast carcinoma, the content of p85β correlated with invasion/metastasis. Thus, the levels of PIK3R2 in breast can indicate potential recurrence of the tumor and support the decision of the need for adjuvant therapy.

Therefore, these studies show that the expression of PIK3R2 is a biomarker for tumor progression, since it is associated with the tumor grade in CC and invasiveness in BC.

### Example 3

### Activation of the PI3K Pathway is Increased in Cells that Express p85β

In order to compare the activities of p85α/p110α and p85β/p110α in vivo, the status of activation of different effectors of the PI3K pathway in cells expressing recombinant p110α (r) and rp85α or rp85 β was examined.

Mouse embryonic fibroblasts (MEF) and NIH3T3, COS-7 and U2OS cells were cultured as described earlier (Marqués M., et al., 2008, Mol Cell Biol., 28: 2803-2814). The cells were transfected with Lipofectamine (Invitrogen). The empty vectors pSG5, pSG5-p85α, -V12Ras and myc-p110α have already been described earlier. The pEGFP-PH-Btk plasmid that encodes the PH domain of the Bruton's tyrosine kinase was ceded by T. Balla (National Institutes of Health, Bethesda, Md.). The pT7/T3-U19 vector that encodes murine p85β was ceded by J. W. G. Janssen (Institute fur Humangenetik, Heidelberg, Germany). p85β was subcloned in pSG5, introducing an hemaglutinin epitope (HA) N-terminal. The p85β ATG codon was replaced by a proline and the HA-tag ATG codon was remained (Quickchange mutagenesis kit; Stratagene). The control siRNA and the siRNA for p85β were from Dharmacon.

For the PI3K cell tests, cells remained inactive between 19 (NIH3T3) or 48 hrs (COS7) without serum; some were treated 10 min in medium containing 10% serum or 50 ng/ml of PDGF (Calbiochem). Extracts were prepared in a TX-100 lysis medium; an IP was carried out with PI3K with the appropriate antibody. For immunofluorescence, cells were fixed in 4% paraformaldehyde in PBS (15 min), and were permeated in PBS with 1% BSA and 0.3% TX-100, they were subsequently blocked with 1% BSA, 10% goat serum and 0.01% TX-100 in PBS (30 min). The cells were visualized using a 60×1.3NA PLOIL objective in an Olympus Fluoview 1000 microscope.

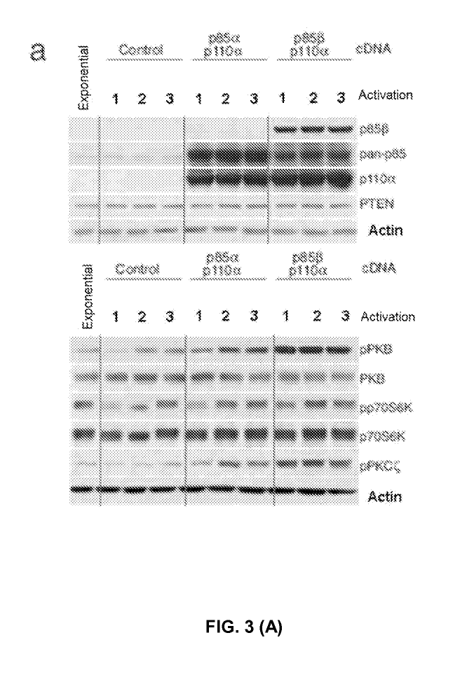

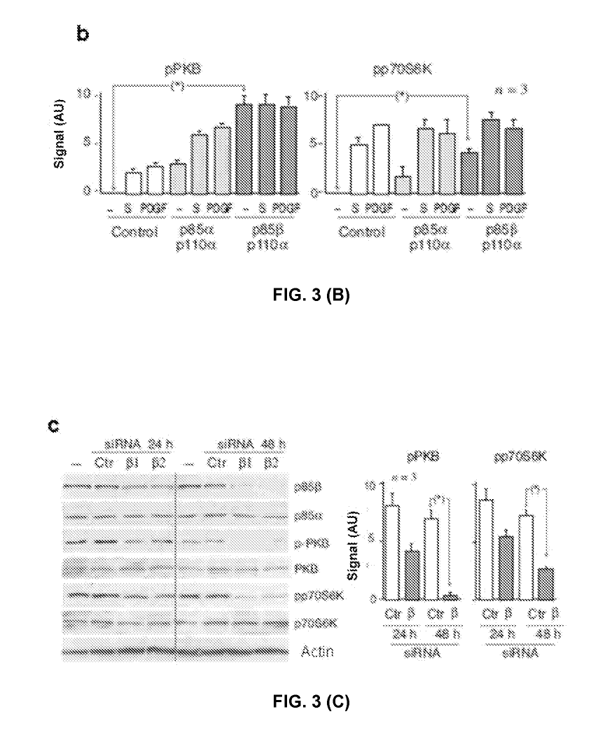

After transfection of the COS-7 cells with the suitable cDNA, the activity of the PI3K molecular targets in extracts of quiescent or activated cells was examined (10 min with medium containing 10% serum or 50 ng/ml of PDGF). p85β appeared associated at a 1:1 ratio with p110α, the same as p85α. The expression levels of p85α and p85β were comparable; and so too was the expression of rp110α (approximately 10 times greater than the endogenous levels) (FIG. 3a). The treatment with PDGF or serum increased the amount of p-PKB, p-p70s6k and p-PKC in control cells; the activation of these effectors was higher in cells expressing higher levels of p110α (FIG. 3a). Moreover, despite the similarity of expression of p85α/p110α and p85β/p110α, the p85β/p110α cells showed a greater activation of the PI3K effectors even in the absence of serum (FIGS. 3a and 3b). A similar analysis in NIH3T3 cells showed a similar result, although the expression of rp110α was only about 2 times greater than the endogenous and the activation of the pathway in the absence of serum was less prominent. Therefore, in vivo, p85β/p110α enhanced activation of the PI3K pathway.

To demonstrate the contribution of p85β to the control of the activation of the PI3K pathway in vivo, the p85β levels were reduced by using siRNA in HeLa cells. The p85β siRNA, but not control siRNA, reduced the levels of p85β and p-PKB and p-p70s6k in the cells (FIG. 3c). This result confirmed the contribution of p85β to the control of the activation of the PI3K pathway in transformed cells.

Thus, the contribution of PIK3R2 to tumor progression suggests that therapies aimed at reducing the expression or action of p85β (such as siRNAs) are useful for cancer treatment

### Example 4

### p85β Increases the Levels of Membrane PIP₃ and Induces Cell Transformation

To confirm the increased activity of p85β/p110α for its physiological substrate PtdIns(4,5)P₂, the in vivo formation of PIP₃ was assessed by co-transfection of p85β/p110α or p85α/p110α with the green fluorescent protein (GFP) fused to the PH domain of Btk, which selectively binds PIP₃. The levels of Btk-PH in the cell membrane in quiescent and treated with serum (10 min) NIH3T3 cells expressing p85β/p110α or p85α/p110α were analyzed.

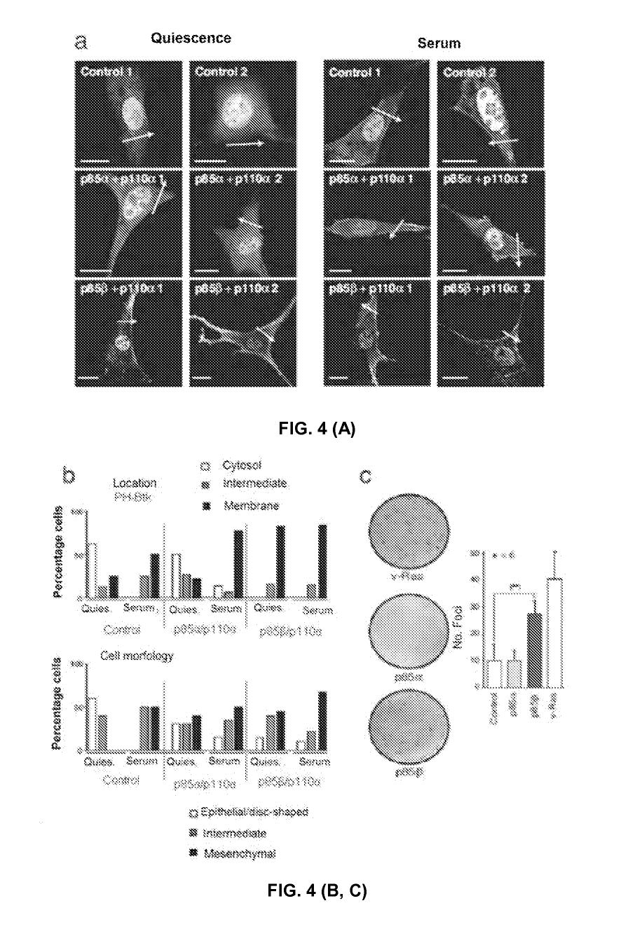

The Btk-PH expression levels were similar in control cells and in the cells transfected with p85β/p110α and p85α/p110α. In control cells, Btk-PH was located in the cytoplasm and in the nucleus (FIG. 4a). The addition of serum produced a relocation of part of the Btk-PH to the cell membrane both in control cells and in p85α/p110α cells. However, in p85β/p110α quiescent cells the majority of Btk-PH was in the membrane, and this fraction increased by adding serum (FIG. 4a). Quantification of fluorescence signal in a high number of samples (n=50) confirmed that this phenotype was general (FIG. 4b). In addition, incubation with serum induced a change from disc-shaped/epithelial to mesenchymal morphology (FIG. 4a). Compared with control cells, the percentage of cells with mesenchymal morphology was slightly increased in p85α/p110 cells since these express higher levels of p110α (FIG. 4b). In addition, a large proportion of the p85β/p110α cells showed this migratory morphology prior to the addition of serum (FIG. 4b). These results indicate that the expression of p85β increases the levels of PIP₃ in the membrane and causes a migratory phenotype.

Due to the effect of p85β on PI3K activity, and the function of PI3K in cell transformation, an assay of foci formation was used to test the ability of p85β to induce transformation. While the expression of p85α does not transform NIH3T3 cells, p85β was capable of inducing foci formation although to a lesser degree than V12-Ras (FIG. 4c).

These results show that tumor progression correlates with alterations in the levels of the regulatory subunits of PI3K. The increase of p85β and the decrease of p85α cause an enrichment of p85β/p110α complexes that have higher affinity for the physiological substrate PtdIns(4,5)P₂. This increases the production of PIP₃ even in the absence of growth factors explaining the transforming capacity of p85β and its role in tumor progression.

These tests show the different effect of p85α and p85β on p110α activity, since when associated to p85β, p110α has a greater binding to its physiological substrate PtdIns (4,5)P₂. The increase in the p85β/p110α complexes results in increased production of PIP₃ and, consequently, in a greater activation of the PI3K effectors: PKB and p70s6k, even in the absence of stimulation, providing the tumor with factor independence for its growth. Finally, the increase of expression of the p85β regulatory subunit is a frequent event in colon and breast carcinomas that increases the PI3K activity in the absence of stimulus. The increase of p85β is, thus, a prognostic factor of tumor progression.

1. Use of the PIK3R2 gene or its expression products for the diagnosis, prognosis and monitoring of cancer.

2. Use of the PIK3R2 gene or its expression products according to claim 1 wherein the cancer is breast or colon cancer.

3. Method for obtaining data useful for the diagnosis, prognosis and monitoring of cancer comprising:
a. obtaining an isolated biological sample comprising tumor cells from an individual,
b. detecting the amount of expression product of the PIK3R2 gene in the isolated biological sample from (a), and
c. comparing the amount detected in step (b) with a reference amount.

4. Method according to claim 3 further comprising:
d. assigning the individual from step (a) to the group of patients with advanced tumor stage when the amount detected in step (b) is greater than the reference amount.

5. Method according to claim 3 wherein the cancer is colon cancer.

6. Method according to claim 3 wherein the cancer is breast cancer.

7. Method according to claim 6 further comprising:
d. assigning the individual from step (a) to the group of patients with invasive cancer when the amount detected in step (b) is greater than the reference amount.

8. Method according to claim 3 wherein the reference amount comes from an isolated biological sample that does not comprise tumor cells.

9. Method according to claim 3 wherein the individual is a mammal.

10. Method according to claim 9 wherein the mammal is a human.

11. Kit for the diagnosis, prognosis and monitoring of cancer comprising the primers, probes or antibodies, or any combination thereof, necessary for detecting the amount of expression product of the PIK3R2 gene.

12. Use of the kit according to claim 11 for the diagnosis, prognosis and monitoring of cancer.

13. Use of the kit according to claim 12 wherein the cancer is breast or colon cancer.

14. Use of at least one inhibitor of the PIK3R2 gene or its expression products for the preparation of a medicament for cancer treatment.

15. Use of at least one inhibitor of the PIK3R2 gene or its expression products according to claim 14 wherein the cancer is breast or colon cancer.

16. Method according to claim 4 wherein the cancer is colon cancer.

17. Method according to claim 4 wherein the reference amount comes from an isolated biological sample that does not comprise tumor cells.

18. Method according to claim 4 wherein the individual is a mammal.
